(19) United States
(12) Patent Application Publication
(10) Pub. No.: US 2003/0188752 A1
(43) Pub. Date: Oct. 9, 2003

(54) Device and method for maintaining an airway in an open condition useful for anesthetized or other breathing difficulty states

(76) Inventors: Christine M. Zirafi, Rocky River, OH (US); Michael J. Cwalina, Independence, OH (US)

Correspondence Address: PEARNE & GORDON LLP, 526 SUPERIOR AVENUE EAST, SUITE 1200, CLEVELAND, OH, 44114-1484, US

(21) Appl. No.: 10/115,855

(22) Filed: Apr. 3, 2002

(51) Int. Cl. A61F 5/56

(52) U.S. Cl. 128/848

(57) ABSTRACT

A device and associated method maintain an airway of a person in an open condition. A first portion of the device is for fixed attachment at a location adjacent to a neck of the person. A second portion of the device is connected to the first portion. The second portion is for engagement with a chin of the person. The second portion is adjustable to move the chin, relative to the first portion while the first portion remains in fixed attachment at the location adjacent to the neck of the person. In one embodiment, the first portion is provided as a collar, and the second portion is provided to be inflatable. The location of the chin relative to the collar portion is changed with change in an inflation volume of the inflatable portion.

### TECHNICAL FIELD

The present invention relates to a medical device, and more particularly, to a device for maintaining an airway of a person in an open condition.

### BACKGROUND OF THE INVENTION

It is essential that an airway of a person remain open such that the person continues with normal respiration. During some states (e.g., unconsciousness or sleep) of the person, it may become difficult for the person to maintain an open airway without assistance. Such states may include a state of anesthetized unconsciousness of the person. Also, such states may include a state during natural sleep associated with sleep apnea. For such example states of the person, obstruction of the airway by tissue may occur.

Often, airway obstruction during such states is associated with relaxation of muscles. In one example, obstruction may occur near the pharynx as a result of the relaxed muscles. Anaesthesiologists are knowledgeable of such issues associated with the administration of general anaesthesia for a surgical procedure. As such, it is common to manually lift the lower jaw during the procedure to aid in maintaining the airway open. The lifting can be associated with a slight extension of the head relative to the thorax. The lifted lower jaw provides for a lifting of the tongue away from the posterior pharyngeal wall to avoid obstruction.

There have been several techniques contemplated that are directed to maintenance of an open airway passage. Some of these techniques include the use of an inserted endotracheal tube. However, such an approach has several drawbacks including most commonly throat irritation. Other approaches include the use of external devices that are attached externally about the neck of a person. With these devices, proper positioning may become an issue.

### SUMMARY OF THE INVENTION

In accordance with one aspect, the present invention provides a device for maintaining an airway passage of a person in an open condition. A first portion of the device is for fixed attachment at a location adjacent to a neck of the person. The second portion of the device is connected to the first portion. The second portion is for engagement with a chin of the person. The second portion is adjustable to move the chin, relative to the first portion while the first portion remains in fixed attachment at the location adjacent to the neck of the person.

In accordance with another aspect, the present invention provides a device for maintaining an airway passage of a person in an open condition. A collar portion of the device extends adjacent to a neck of the person. An inflatable portion of the device extends from the collar portion. The inflatable portion is engageable with a chin of the person. A location of the chin relative to the collar portion is changed with change in an inflation volume of the inflatable portion.

In accordance with yet another aspect, the present invention provides a method of maintaining an airway passage of a person in an open condition. A first portion of a device is fixed at a location adjacent to a neck of a person. A second portion of the device is connected to the first portion and is in engagement with a chin of the person. The second portion is adjusted to move the chin relative to the first portion while the first portion remains at the location adjacent to the neck of the person.

### DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is a schematic illustration of an example of the present invention in position on a person; and

FIG. 2 is an illustration similar to FIG. 1, but showing the device a different adjustment.

### DESCRIPTION OF EXAMPLE EMBODIMENT

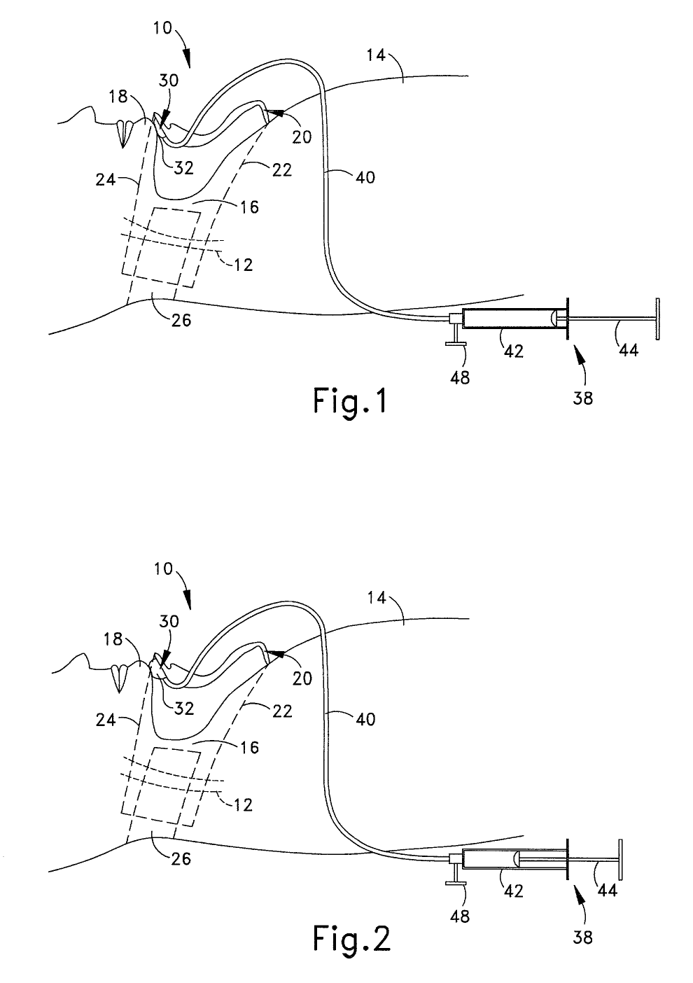

An example of a device 10 for maintaining an airway passage 12 (schematically shown) of a person 14 (e.g., a patient) in an open condition is shown in FIG. 1. The device 10 is intended for use about a neck 16 of the person 14. Specifically, the device 10 is for engagement with a chin 18 of the person 14.

A first portion 20 of the device 10 is for location adjacent to the neck 16, and in one example is provided as a padded neck collar portion. Hereinafter, the first portion 20 is referred to as the collar portion 20. The collar portion 20 is shown partially in phantom to permit easy visualization of other structure and relationships to be described below.

The collar portion 20 extends substantially about the neck 16 of the person 14. A lower edge 22 of the collar portion 20 rests upon the upper thorax of the person 14. An upper edge 24 of the collar portion 20 extends proximate to the chin 18 and jaw of the person 14.

The collar portion 20 has any suitable construction that provides a substantially rigid platform. As such, the construction of the collar portion 20 may include a dense closed-cell foam with a fabric covering. The collar portion 20 may also include a rigid plastic infrastructure. Also, the construction may be open or closed.

Associated with the collar portion 20 is an attaching mechanism 26 (shown in phantom). In one example, the attaching mechanism 26 is part of the collar portion 20. In the illustrated example, the attaching mechanism 26 is provided an extending strap that includes a hook and loop fastening arrangement. Such hook and loop fastening is commonly referred to as Velcro (TM). The strap is attached to one side (e.g., left side) of the collar portion 20. The strap contains one portion (e.g., hook portion) of the hook or loop arrangement, whereas the complimentary portion (e.g., loop portion) of the arrangement is located on the other side (e.g., right side) of the collar portion 20. It is to be appreciated that the collar portion 20 and the associated attaching mechanism 26 may have different constructions.

With the collar portion 20 placed about the neck 16 of the person 14, the strap is wrapped around the posterior of the neck 16 and accordingly fastened. This generally fixes the collar portion 20 relative to the person 14. Specifically, the lower edge 22 of the collar portion 20 rests firmly on the upper thorax of the person 14.

A second portion 30 of the device 10 is located adjacent to the lower surface of the chin 18. The second portion 30 is inflatable (FIG. 2) via provision of inflation fluid (e.g., air), and extends upon inflation. At least during some point during inflation of the second portion 30, the second portion presses against the chin 18. Hereinafter, the second portion 30 is referred to as the inflatable portion 30.

The inflatable portion 30 is mounted on the collar portion 20, at least on one side of the inflatable portion, by any suitable attaching means. Accordingly, upon inflation of the inflatable portion 30, a distal surface 32 of the inflatable portion extends away from the point of attachment with the collar portion 20. As such, the inflatable portion 30 provides the pressing or pushing force upon the chin 18, as shown in FIG. 2. Such pushing provides a lifting movement of the chin 18. The movement of the chin 18 is relative to the collar portion 20 which remains in fixed attachment adjacent to the neck 16. The lifting of the chin 18 helps maintain the airway passage 12 of the person 14 in an open condition. In one example, the movement may be up to approximately two inches of travel distance. It is to be appreciated that other movement amounts are possible.

The inflatable portion 30 may have any suitable construction to accomplish the inflation and pressing movement of the chin 18. In one example, the inflatable portion 30 is an air bladder made of latex. However, it is to be appreciated that other constructions and moving mechanisms are contemplated. As such, the illustrated example set forth herein is not intended to be a limitation on the boundaries of the present invention.

In the illustrated example, the inflation fluid that is utilized to inflate the inflatable portion 30 is air. Also, in the example, inflation fluid is supplied to the inflatable portion 30 via a syringe 38 as a supply portion of the device 10. Further, in the example, the syringe 38 is located at a distance from the inflatable portion 30, and is connected to the inflatable portion via a tubing 40.

The tubing 40 extends from the inflatable portion 30 out from the collar portion 20 and to the syringe 38. The connection of the tubing 40 to the inflatable portion 30 may have any suitable construction and configuration. Also, the connection and cooperation of the tubing 40 as it extends from the collar portion 20 may have any suitable construction and configuration.

Turning to the syringe 38, the syringe includes a housing 42 that encloses a volume of fluid (e.g., air). A plunger 44 is movable relative to the housing 42 to force the inflation fluid from the housing 42 through the tubing 40 and to the inflatable portion 30. The movement of the plunger 44 is via manual actuation of the syringe 38. However, it is to be appreciated that a different inflation fluid (e.g., air) supply means may be utilized.

In one example, it may be desirable to maintain the inflation level of the inflatable portion 30. As such, a stopcock valve 48 may be employed in-line with the syringe 38 and the tubing 40. The valve 48 is manually actuatable to permit or prevent passage of the air fluid to/from the inflatable portion 30.

The level to which the chin 18 is moved and lifted is adjustable while the collar portion 20 remains in fixed attachment at a location about the neck 16 of the person 14. Specifically, the adjustment is provided by adjusting the amount of air fluid within the inflatable portion 30. The inflation fluid may be added to, or subtracted from, the inflatable portion to provide fine adjustment of the lifting movement provided to the chin 18.

Accordingly, in order to maintain an airway passage of a person in an open position, the collar portion 20 is placed about the neck 16 and the attaching mechanism 26 (e.g., the strap) is fastened to secure the collar portion 20 in place. The syringe 38 is actuated to force inflation fluid into the inflatable portion 30 and lift the chin 18 of the person 14. The inflation is continued until the desired lifting is provided to the chin 18. If necessary, some amount of inflation may be withdrawn from the inflatable portion 30 via the syringe 38 to obtain a desired level of chin lifting.

The present invention is applicable to maintain an airway passage of a person in an open condition during an application of general anaesthesia. The present invention may also be applicable to other scenarios including the use to maintain an open airway passage condition during sleep apnea or the like.

The device of the present invention may be used in the transport of a patent in the field. Also, the device of the present invention may be used to facilitate external bagging of a patient (e.g., forcing air into the patient) with an Am-Bu device. Still further, the device of the present invention may be used with an oral airway device for a patient that cannot be intubated in the field by a paramedic. Such use would provide for ease in bagging the patient while en route to a hospital.

From the above description of the invention, those skilled in the art will perceive improvements, changes and modifications. Such improvements, changes and modifications within the skill of the art are intended to be covered by the appended claims.

Having described the invention, the following is claimed:

1. A device for maintaining an airway of a person in an open condition, the device including:
a first portion for fixed attachment at a location adjacent to a neck of the person; and
a second portion connected to the first portion and for engagement with a chin of the person, said second portion being adjustable to move the chin relative to the first portion while the first portion remains in fixed attachment at the location adjacent to the neck of the person.

2. A device as set forth in claim 1, wherein the second portion includes an inflatable bladder.

3. A device as set forth in claim 2, including a third portion for supplying inflation fluid to the bladder.

4. A device as set forth in claim 3, wherein the inflation fluid is air.

5. A device as set forth in claim 3, wherein the third portion includes a syringe.

6. A device as set forth in claim 1, wherein the first portion includes a collar.

7. A device as set forth in claim 6, wherein the first portion includes an attachment strap.

8. A device as set forth in claim 1, wherein the movement of the chin is a lifting movement of the chin.

9. A device for maintaining an airway of a person in an open condition, the device including:
a collar portion extending adjacent to a neck of the person; and
an inflatable portion extending from the collar portion and engageable with a chin of the person, location of the chin relative to the collar portion being changed with change in an inflation volume of the inflatable portion.

10. A device as set forth in claim 9, wherein the inflatable portion includes a bladder.

11. A device as set forth in claim 10, including a supply portion for supplying inflation fluid to the bladder.

12. A device as set forth in claim 11, wherein the inflation fluid is air.

13. A device as set forth in claim 11, wherein the supply portion includes a syringe.

14. A device as set forth in claim 9, wherein the change of location of the chin includes a lifting movement of the chin.

15. A method of maintaining an airway of a person in an open condition, the method including:
fixing a first portion of a device at a location adjacent to a neck of the person; and
adjusting a second portion of the device, the second portion being connected to the first portion and in engagement with a chin of the person, to move the chin relative to the first portion while the first portion remains at the location adjacent to the neck of the person.

16. A method as set forth in claim 15, wherein the step of fixing a first portion includes fixing a collar, the collar being the first portion, and the step of adjusting a second portion includes adjusting inflation volume in an inflatable portion, the inflatable portion being the second portion.

17. A method as set forth in claim 16, wherein the inflatable portion includes a bladder.

18. A method as set forth in claim 17, wherein the step of adjusting a second portion including supplying inflation fluid to the bladder.

19. A method as set forth in claim 18, wherein the step of supplying inflation fluid includes supplying the inflation fluid from a syringe.

20. A method as set forth in claim 15, wherein the movement of the chin is a lifting movement of the chin.
